(19) United States
(12) Patent Application Publication
(10) Pub. No.: US 2024/0299761 A1
(43) Pub. Date: Sep. 12, 2024

(54) METHOD AND SYSTEM FOR MACHINE LEARNING ADJUSTMENT OF CHEMICAL COMPOSITION OF COLD ATMOSPHERIC PLASMA JET

(71) Applicant: The George Washington University, Washington, DC (US)

(72) Inventors: Michael KEIDAR, Washington, DC (US); Li LIN, Washington, DC (US); Taeyoung LEE, Washington, DC (US)

(21) Appl. No.: 18/551,994

(22) PCT Filed: Mar. 22, 2022
(86) PCT No.: PCT/US2022/021397

§ 371 Date: Sep. 22, 2023

Related U.S. Application Data
(60) Provisional application No. 63/164,968, filed on Mar. 23, 2021.

(51) Int. Cl. A61N 1/44; A61N 1/02; A61N 1/08; G01J 3/443; G16H 50/20

(52) U.S. Cl. CPC A61N 1/44; A61N 1/025; A61N 1/08; G01J 3/443; G16H 50/20

(57) ABSTRACT

A method and system of real-time determination of control parameters for generating a plasma jet with a specific chemical composition is disclosed. The system includes a controller coupled to a gas source and a voltage source to generate a plasma jet having the desired chemical composition via control parameters for the gas and voltage. An optical emission spectroscopy sensor detects spectral data from the plasma jet. A diagnostic neural network module is trained to output chemical compositions and energy distribution of gasses of the gas source from an input of spectral data. The detected spectral data is input to the diagnostic neural network. A control neural has an input of the chemical compositions and energy distribution output by the diagnostic neural network and an output of the control parameters. The control neural network is trained via chemical compositions output from the diagnostic neural network and the desired chemical composition.

### PRIORITY CLAIM

The present application claims priority to U.S. Provisional Patent Application Ser. No. 63/164,968, filed Mar. 23, 2021. The entirety of this application is hereby incorporated by reference.

### STATEMENT REGARDING FEDERALLY SPONSORED RESEARCH

This invention was made with government support under 1919019 awarded by National Science Foundation. The government has certain rights in the invention.

### TECHNICAL FIELD

The present invention relates generally to cold atmospheric plasma, and more particularly, using machine learning analysis to determine an optimal chemical composition for generating a cold atmospheric plasma jet.

### BACKGROUND

Low-temperature plasmas offer an unmatched, unique platform to affect and control biological processes. Plasma based engineering of biological processes is a new multi-disciplinary field that converges engineering, physics, chemistry, materials science, and biology. Plasma based engineering focuses on the interaction of the reactivity produced by atmospheric pressure plasma with soft biological matter and systems (e.g., liquids, cells, tissues, water, food, plants, agricultural products).

Cold atmospheric plasma (CAP) is a type of thermal non-equilibrium plasma at atmospheric pressure. The ionization degree of CAP is usually about Oct. 8, 2010-5. The plasma is in thermal non-equilibrium in that only the mean temperature of electrons is elevated to 5-10 eV while those for other heavy particles are near room temperature. Therefore, the cold plasma naturally becomes a source of complex chemistry and electromagnetic radiation for biomedicine and material processing without any thermal damages. In recent years, more biomedical applications of CAP have been discovered, including bacteria and biofilm sterilization, wound healing, cancer therapy, and virus sterilization which includes the deactivation of SARS-COV-2, the very virus causing the global COVID-19 pandemic. A simple CAP treatment can cause drastic inactivation on Gram-positive and Gram-negative bacteria not only in colonies but also in biofilm. As a promising anti-cancer modality, CAP selectively kills many cancer cell lines but while just causing limited side effects on normal cell lines in vitro. More importantly, CAP also shows strong growth inhibition on tumors in vivo by a simple treatment even in a transdermal way. Reactive species are the key players in CAP to trigger the observable biological effects. Many of them, particularly hydrogen peroxide (H2O2), hydroxyl radical (·OH), superoxide anion (O2-), singlet oxygen (¹O2), are toxic to microorganisms and cancer cells by causing strong damage to DNA, RNA, proteins, and phospholipid.

Cancer is one of the most fatal diseases in modern medicine. For example, Melanoma is a common skin and mucosa tumor, with a high mortality rate so far. Melanoma is also one of the fastest growing malignant tumors. The current therapy is mainly based on surgical treatment, radiotherapy, and chemotherapy. Such previous modalities tend to affect the growth of cancer cells via directly causing apoptosis or other cell deaths. For example, cold atmospheric plasma is a potential method to treat melanoma based on its abundant reactive species components. The CAP treatment kills cancer cells through the direct transportation of these reactive species into the extracellular environment. In one study, CAP-treated pancreatic adenocarcinoma cells (PA-TU-8988T) could quickly enter into a specific state, in which cells were very sensitive to the cytotoxicity of reactive species, such as H2O2 and NO2. Such activation is partially due to a physical effect such as the electromagnetic field.

The cold atmospheric plasma (CAP) is usually a capacitive-coupled atmospheric plasma guided with a noble gas. CAP-based treatment relies on a complex plasma-liquid multiphase chemistry near and on interfaces. Taking a CAP jet as an example, during a streamer propagation, energetic electrons dissociate bonds such as O2+e=>2O+e and N2+e=>2N+e. The atomic Nitrogen and Oxygen thus generate NO and NO2. The atomic Oxygen is highly active and is responsible to produce O3 for biomedical sterilization and other reactive oxygen species (ROS), such as the H2O2, for oxidic stress. The aqueous solution is the main hydrogen element provider for ROS, along with the humidity in the air. Therefore, these key species of biomedical applications appear at the plasma-liquid interface. In the view of pharmacology, plasma medicine must show a significant effect with limited side effects during treatment. In other words, since only a part of reactive species are the active pharmaceutical ingredients (API), the compositions of others should be limited in CAPs. Considering the complex plasma chemistry at the plasma-target interface a well-designed control mechanism is required to optimize the CAP chemistry for specific purposes.

The control of plasma chemistry is a two-step process. The first step is a real-time diagnostic of plasma chemistry. The second step is to optimize the chemical composition by controlling the plasma generator. First, real-time measurement of the species composition is usually based on active measurements such as laser-induced fluorescence (LIF). However, these methods can only provide the densities of certain species, depending on the wavelength of the incoming laser, rather than an entire picture of all the important reactive species, such as the molecular beam mass spectroscopy. Also, LIF is an active detection method that requires a tunable laser if various species are accessed. Similar to other absorption and scattering methods such as the laser Thomson scattering (LTS), the active measurement means a trade-off between altering the plasma and a low signal-to-noise ratio. In contrast, passive detections based on the spontaneous emissions of the plasma such as the optical emission spectroscopy (OES) do not suffer from such a dilemma. However, the main disadvantage of passive methods is the indirect relationship between the spectrum and compositions, considering dozens of species involving hundreds to thousands of collisions (chemical reactions), with dynamic and unique rate coefficients for each collision. As such, it is virtually impossible to perform a reverse computation by regular analytical and numerical methods due to its complexity.

The major biomedical effects of CAP treatment are its reactive oxygen species such as reactive oxygen nitrogen species (RONS), although physical effects such as thermal, UV, and microwave emissions are also considered in some recent research works. Comparing with the physical dose applications prementioned, the chemical dose is a more complicated system to control, due to its higher dimension of the action space, considering each species density is a dimension.

Currently known methods will alter the desired composition for specific applications. However, these methods rely on either simulations of the composition or active methods which may alter the plasma during the measurements. For example, existing systems use a complex hardware system with simple computation of compositions. Such systems are not flexible enough to account for other disturbances that may affect the plasma jet. During an in vivo treatment such as surgery, such disturbances can be common. Also, the conditions of patients are different from one another. This means that the treatment goal, the chemical environment, and the boundary condition can be different and dynamic.

There is a need for a reverse computation method using an artificial neural network (ANN) to achieve passive and real-time plasma chemical diagnostics for a cold atmosphere plasma system. There is a further need to apply a machine-learning (ML) method to achieve real-time control of plasma chemistry in a cold atmosphere plasma system. There is a further need for a trained control neural network that can provide control parameters to adjust different species in a cold atmospheric plasma system.

### SUMMARY

One disclosed example is a system for treatment of a target area. The system includes a plasma jet emitter emitting a plasma jet at the target area. The system includes a gas source providing a gas composition to the plasma jet emitter and a voltage source coupled to an electrode in the plasma jet emitter. A controller is coupled to the gas source and the voltage source to control the plasma jet emitter to generate a plasma jet having a desired chemical composition via control parameters for the gas source and the voltage source. An optical emission spectroscopy sensor detects spectral data from the plasma jet. A diagnostic neural network module is trained to output chemical compositions and energy distribution of gasses of the gas source from an input of spectral data from the optical emission spectroscopy sensor. The detected spectral data is input to the diagnostic neural network. A control neural network module is coupled to the controller and the diagnostic neural network. The control neural network has an input of the chemical compositions and energy distribution of the gasses of the gas source output by the diagnostic neural network and an output of the control parameters. The control neural network is trained via chemical compositions output from the diagnostic neural network and the desired chemical composition.

A further implementation of the example system is an embodiment where the system further includes a database coupled to the controller. The database stores trained control neural network data for chemical compositions including the desired chemical composition. Another implementation is where chemical composition of the plasma jet includes at least one of a reactive oxygen or nitrogen species. Another implementation is where the gasses of the gas source include a noble gas, oxygen, and nitrogen. Another implementation is where the system includes a magnetic field generator controlled by the controller and directing a magnetic field at the target area. The control parameters include the strength of the magnetic field. Another implementation is where the spectral data is stored in the database to update a training set for the diagnostic neural network. Another implementation is where the training of the diagnostic neural network includes comparison of spectral data determined by a chemical simulation of the chemical composition with the results of the chemical composition determined by the diagnostic neural network based on the spectral data. Another implementation is where the training of the control neural network compares control parameters output from the control neural network and control parameters associated with a simulation of the desired composition. Another implementation is where the training of the diagnostic neural network and control neural network uses a gradual mutation algorithm (GMA) to create multiple mutation neural networks providing unique outputs and selects the mutation neural network with a lowest error for a next iteration of training. Another implementation is where the chemical composition maximizes the summation of densities of OH, HO2, H2O2, and OH ions as the active pharmaceutical ingredients (API) and the treatment is apoptosis of cancer cells in the target area. Another implementation is where the chemical composition maximizes the summation of the densities of NO and its ions and the treatment is healing of a wound in the target area. Another implementation is where the chemical composition maximizes the summation of the densities of O3 and its ions and the treatment is sterilizing the target area.

Another disclosed example is a system to train a control neural network to provide control parameters to generate a plasma jet with a selected chemical composition. The system includes an optical emission spectroscopy sensor to detect spectral data from a plasma jet generated with source gasses and voltage. A diagnostic neural network is trained to output the chemical composition of the plasma jet from input data including the detected spectral data. The diagnostic neural network is trained by comparing the chemical composition determined from the detected spectral data and the results of a chemical simulation based on the selected source gasses and voltage. The control neural network provides an output of control parameters from an input of a chemical composition provided by the trained diagnostic neural network. The control neural network is trained by comparing the output control parameters with the control parameters associated with the selected chemical compositions from a chemical simulation.

A further implementation of the example system is an embodiment where the gasses of the gas source include a noble gas, oxygen, and nitrogen. Another implementation is where the training of the diagnostic neural network includes comparison of spectral data determined by a chemical simulation of the chemical composition with the results of the chemical composition determined by the diagnostic neural network based on the spectral data. Another implementation is where the training of the control neural network compares control parameters output from the control neural network and control parameters associated with a simulation of the desired composition. Another implementation is where the training of the diagnostic neural network and control neural network uses a gradual mutation algorithm (GMA) creating multiple mutation neural networks providing unique outputs and selects the mutation neural network with a lowest error for a next iteration of training. Another implementation is where the chemical composition maximizes the summation of densities of OH, HO2, H2O2, and OH ions as the active pharmaceutical ingredients (API) and the treatment is apoptosis of cancer cells in the target area. Another implementation is where the chemical composition maximizes the summation of the densities of NO and its ions and the treatment is healing of a wound in the target area. Another implementation is where the chemical composition maximizes the summation of the densities of O3 and its ions and the treatment is sterilizing the target area.

Another disclosed example is a method of generating a plasma jet having a specific chemical composition. A desired chemical composition is selected for the plasma jet. The plasma jet is generated and spectral data from the plasma jet is detected. The spectral data I input to a diagnostic neural network to output the chemical composition of the plasma jet. The diagnostic neural network is trained by spectral data from compositions of plasma jets. A set of control parameters is determined via a control neural network trained via chemical compositions output by a diagnostic neural network. The control parameters are applied to a gas source and a voltage generator to adjust the plasma jet via a controller. The generated plasma jet is directed to a target area for treatment.

A further implementation of the example method is an embodiment where the control neural network is programmed from trained control neural network data for a plurality of chemical compositions including the desired chemical composition stored in a database. Another implementation is where the chemical composition of the plasma jet includes at least one of a reactive oxygen or nitrogen species. Another implementation is where the gasses of the gas source include a noble gas, oxygen, and nitrogen. Another implementation is where the method includes directing a magnetic field at the target area via a magnetic field generator controlled by the controller. The control parameters include the strength of the magnetic field. Another implementation is where the method includes storing the spectral data in a database to update a training set for the diagnostic neural network. Another implementation is where the training of the diagnostic neural network includes comparison of spectral data determined by a chemical simulation of the chemical composition with the results of the chemical composition determined by the diagnostic neural network based on the spectral data. Another implementation is where the training of the control neural network compares control parameters output from the control neural network and control parameters associated with a simulation of the desired composition. Another implementation is where the training of the diagnostic neural network and control neural network uses a gradual mutation algorithm (GMA) to create multiple mutation neural networks providing unique outputs and selects the mutation neural network with a lowest error for a next iteration of training. Another implementation is where the chemical composition maximizes the summation of densities of OH, HO2, H2O2, and OH ions as the active pharmaceutical ingredients (API) and the treatment is apoptosis of cancer cells in the target area. Another implementation is where the chemical composition maximizes the summation of the densities of NO and its ions and the treatment is healing of a wound in the target area. Another implementation is where the chemical composition maximizes the summation of the densities of O3 and its ions and the treatment is sterilizing the target area.

Another disclosed example is a method of training a control neural network to output control parameters to generate a plasma jet with a specific chemical composition. Control parameters for voltage and gasses are applied to generate different plasma jets. Spectral data from the plasma jets are detected via an optical emission spectroscopy sensor. A diagnostic neural network is trained to output the resulting chemical composition from the plasma jets based on an input of the detected spectral data. The training includes comparing the spectral data input to the diagnostic neural network and the spectral data produced by a chemical simulation. A chemical composition of the plasma jet determined by the trained diagnostic neural network is provided to train the control neural network to output control parameters for generating a plasma jet with the specific chemical composition.

A further implementation of the example method is an embodiment where the gasses of the gas source include a noble gas, oxygen, and nitrogen. Another implementation is where the training of the diagnostic neural network includes comparison of spectral data determined by a chemical simulation of the chemical composition with the results of the chemical composition determined by the diagnostic neural network based on the spectral data. Another implementation is where the training of the control neural network compares control parameters output from the control neural network and control parameters associated with a simulation of the desired composition. Another implementation is where the training of the diagnostic neural network and control neural network uses a gradual mutation algorithm (GMA) to create multiple mutation neural networks providing unique outputs and selects the mutation neural network with a lowest error for a next iteration of training. Another implementation is where the chemical composition maximizes the summation of densities of OH, HO2, H2O2, and OH ions as the active pharmaceutical ingredients (API) and the treatment is apoptosis of cancer cells in the target area. Another implementation is where the chemical composition maximizes the summation of the densities of NO and its ions and the treatment is healing of a wound in the target area. Another implementation is where the chemical composition maximizes the summation of the densities of O3 and its ions and the treatment is sterilizing the target area

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of embodiments together with reference to the accompanying drawings.

FIG. 1 is a block diagram of an example system that trains a control neural network to maximize gain of certain chemical compositions and control a cold atmospheric plasma generator;

FIG. 2 is a block diagram of an example plasma generator for training the system in FIG. 1;

FIG. 3A is a diagram showing the process of training the diagnostic neural network in FIG. 1;

FIG. 3B is a diagram showing the process of training the control neural network in FIG. 1;

FIG. 4 is a block diagram of a treatment system that uses the trained control neural network to generate a plasma jet for treatment;

FIG. 5 is a diagram of an example plasma jet and the determination of chemical compositions in different regions of the plasma jet via an OES probe;

FIG. 6A is a plot of the errors based on different training iterations for the diagnostic neural network in FIG. 1;

FIG. 6B is a plot of the errors of the testing results of the trained diagnostic neural network in FIG. 1;

FIGS. 7A-7B are plots of the normalized intensity of different test samples plotted in FIG. 6B;

FIG. 8 is an example of the species composition output based on the trained diagnostic neural network in FIG. 1 for two test samples;

FIG. 9A is a graph plotting the prediction of spatial resolution for different species from two of the test samples in FIG. 6B;

FIG. 9B is a distribution plot of the spatially resolved mean electron temperature over time from two of the test samples in FIG. 6B;

FIG. 9C is a series of plots of different species densities based on the test samples;

FIG. 10A is set of graphs that show the convergence of the gain functions for different selections of species in the process of training the control neural network and the resulting outputs from the trained control neural network from the test samples;

FIG. 10B shows the outputs of the trained control neural network; and

FIG. 11 is a flow diagram of the process of controlling a cold atmospheric jet performed to optimize a selected species.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

This disclosure is directed toward an example cold atmospheric plasma jet generator that is controlled by a neural network determination of the complex chemical kinetic schemes of reactive oxygen and nitrogen species in the plasma jet. The optimization of the plasma, such as for medicine (in the form of the specific species) may be performed for specific biomedical purposes to maximize the active pharmaceutical ingredients and minimize other chemical compositions. The disclosed example plasma jet control system incorporates a general method of passive plasma chemical diagnostics and optimization in real-time. Based on spontaneous emission spectroscopy, a first diagnostic artificial neural network provides the gas chemical compositions of a plasma jet along with other information such as the temperatures of the compositions. The composition information further passes through a second control neural network which outputs adjustments of external control parameters including the energy, gas injections, and extractions to optimize the plasma chemistry of the cold atmospheric plasma jet to produce the desired chemical species.

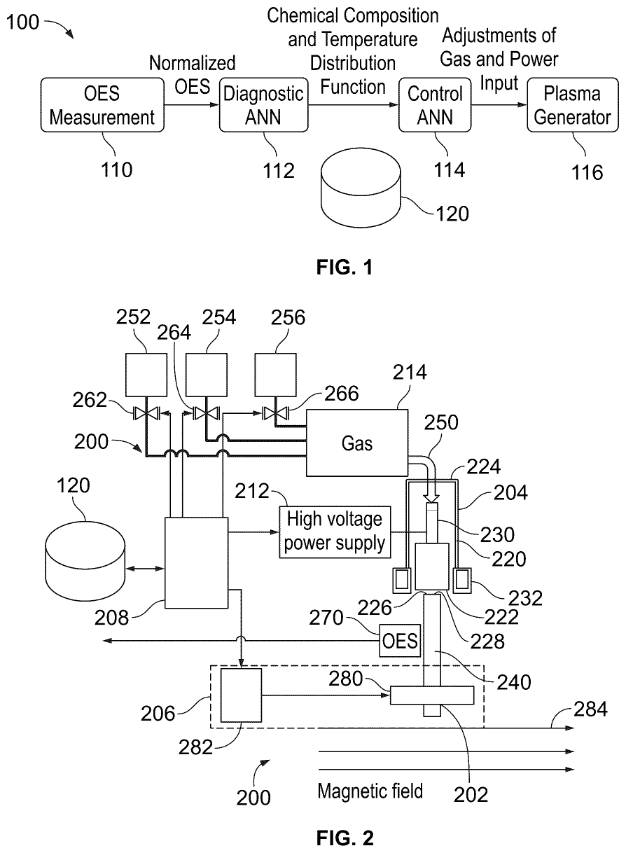

FIG. 1 shows a block diagram of an example training system 100 that employs machine learning to diagnose the plasma chemistry of a plasma jet and train a neural network to adjust the plasma jet control parameters to optimize selected species in the plasma jet in real-time. The system 100 includes an optical emission spectroscopy (OES) measurement module 110, a diagnostic neural network module 112, a control neural network module 114, and a plasma generator module 116. In this example, the machine learning or neural network modules 112 and 114 include artificial neural networks (ANN) that may be executed by any suitable processing device. The method of determining real-time plasma chemistry for the plasma generator 116 relies on real-time OES sensor data from the OES measurement module 110. Plasma jets are generated using different gas flows and voltages to generate a data set to train the neural network of the diagnostic neural network module 112 to output species composition based on input OES sensor data. The OES sensor data produces normalized spectra data that is continuously input into the trained diagnostic neural network module 112. The diagnostic neural network module 112 produces a plasma chemical composition list and energy distributions for each species based on the training data. Once trained, the composition list and distributions output from the trained diagnostic neural network module 112 are input to train the control neural network module 114. The control neural network module 114 outputs control parameters for adjustments of the gas input and power input for the plasma generator module 116 to produce a plasma jet with a desired composition.

The system 100 includes a database 120 that stores training data sets for training the neural network modules 112 and 114. The database 120 may be stored in a separate database managed by a database server or in a suitable processor chipset in a controller. The database 120 may also store reference data such as chemical compositions, chemical simulations, OES data, and resulting species created in the plasma jet that may be used during the training process. The database 120 may also store output data from compositions and resulting control parameters from subsequent actual operation of the plasma generator module 116 to further refine the trained models. The database 120 may also store trained models for the control neural network module 114 for output of different compositions of gasses, different gasses, and desired optimal results that may be used for operation of the plasma generator module 116.

FIG. 2 is a block diagram of the plasma generator module 116 in FIG. 1 that is used to collect data for training and testing the diagnostic neural network module 112. The plasma generator module 116 may be part of a test system 200 that subjects a target area 202 to a cold atmospheric plasma jet 240 for purposes of spectral data collection for the training data set used to train the diagnostic learning module 112. The system 200 includes a cold atmospheric plasma emitter device 204 and an optional magnetic field generator 206. A controller 208 controls the parameters such as gas mixture and voltage strength that generate the plasma jet 240.

The cold plasma emitter device 204 includes a power supply 212, a gas supply 214, and a delivery mechanism 220. In this example, the delivery mechanism 220 is an elongated syringe having a main body 222. The body 222 may be made of glass or a rigid plastic, but also can be made of a flexible material. A proximal end of the body 222 is sealed via a sealing plug 224 and an opposite distal end has a discharge area 226. The distal discharge end 226 of the syringe body 222 has a narrowed neck and a distal opening or nozzle 228. A central electrode 230 is located at the center of the body 222 at the interior of the body 222 at the central longitudinal axis of the syringe 220. The central electrode 230 enters the syringe 220 at the sealed proximal end of the body 222 and extends the length of the body 222 to approximately the discharge end 226. The sealing plug 224 (such as rubber) is located over the open end of the syringe 220 to prevent the gas from escaping from the inside of the syringe 220. The electrode 230 is entirely surrounded by insulation except at its distal end, which is exposed and in contact with gas and plasma. The insulation allows the power to be focused at the exposed distal end to lead to the discharge at the end. The central electrode 230 and surrounding insulation, has a proximal end that extends to the outside of the syringe 220 through an opening in the plug 224. The plug opening forms a friction fit with the insulation, so that gas does not escape from the syringe 220. The central electrode 230 is positioned inside the body 222 of the syringe 220, except for the portion of the proximal end of the electrode 230 that extends into and through the plug.

In this manner, the plug opening holds the electrode 230 and insulation in position within the syringe 220, with the distal end of the electrode 230 facing the distal nozzle 228 of the syringe body 222. In addition, an annular outer ring electrode 232 is located about a portion of the narrow neck at the outside of the syringe 220. The electrodes 230 and 232 are high voltage electrodes. The central electrode 230 may be, for instance, a wire, and the insulation can be a ceramic insulation. The high voltage power supply 212 is electrically connected to the electrodes 230 and 232 and provides a high voltage supply to the electrodes 230 and 232 through cables. The controller 208 regulates the voltage and frequency that is applied to the central electrode 230 and the ring electrode 232 and therefore controls the intensity of the plasma jet 240 emitted by the nozzle 228.

The gas supply 214 is in gas communication with the delivery device 220 through a supply tube 250. The supply tube 250 is connected to a port located on the plug 224 of the syringe 220. The supply tube 250 may also be connected to the syringe 220 through an adapter. The gas supply 214 may include a chamber that can be pressurized, so that gas travels through the supply tube 250 into the inside space of the syringe body 222. A separate gas controller (not shown) may be provided to control the flow rate of the gas in the supply tube 250, or the gas controller may be integrated with the controller 208. The gas then continues through the syringe 220 and exits the syringe 220 through the neck and nozzle 228 at the discharge end as the jet or stream flow 240.

In this example, the controller 208 may control different three gas supplies 252 and 254 that provide three gases that are combined in the gas supply 214. In this example, the gas supply 252 contains a noble gas such as helium, the gas supply 254 contains a first type of gas for producing reactive species such as oxygen (O2), and the gas supply 256 contains a second type of gas for producing reactive species such as nitrogen (N2). The gas flow from the gas supply 252 may be regulated by the controller 208 controlling a valve 262. The gas flow from the gas supply 254 may be regulated by the controller 208 controlling a valve 264. The gas flow from the gas supply 256 may be regulated by the controller 208 controlling a valve 266. In this manner, the controller 208 may vary the gas composition from the gas supply 214 to generate plasma jets of different chemical compositions to train the diagnostic neural network module 112 and the control neural network module 114 in FIG. 1. It is to be understood that there may be multiple gas supplies with different gasses to be combined and controlled via the controller 208 and the system 100 in FIG. 1. As will be explained, a composition may be determined for optimization of different reactive species based on the gas type, composition, and intended effect in treatment.

As the gas enters the discharge area at the nozzle 228 and the neck of the syringe 220, the electrodes 230 and 232 excite the gas, thereby ionizing the gas to form a cold plasma jet. In this example, the gas from the gas supply 214 is composed by the controller 208 based on settings for gas composition and power for the electrodes 230 and 232. Thus, the gas is discharged out of the distal nozzle 228 of the syringe 220 in the form of a cold plasma jet 240. The cold plasma jet or stream flow 240 diffuses over time. In accordance with this example, the plasma may be provided at a flow rate of 10-17 liters per minute, with the discharge voltages between 4 kV and 12 kV and a discharge frequency of 12.5 KHz in accordance with control parameters provided by the trained control neural network. Of course flow rate, discharge voltages and discharge frequency of the plasma jet 240 may be adjusted to different ranges and values. The control parameters for the flow rate, individual gas composition, and voltage and frequency may be adjusted based on the outputs of the control neural network. The actual inputs of flow rate, voltage supply, and frequency as well as gas composition supplied by the gas source may result in plasma jets of different chemical composition that emit optical emission spectra (OES) data for the training set for the diagnostic neural network of the diagnostic neural network module 112.

It should be apparent, however, that other suitable control parameters may be utilized such as the magnetic field strength if a magnetic field is generated by the optional magnetic field generator 206. The actual composition of gas species in the plasma jet stream 240 is determined based on spectral data sensed by an optical emission spectroscopy sensor 270 that provides the detected spectral data to the OES measurement module 110 in FIG. 1. The spectral data indicates the composition and thus different species in the plasma jet stream 240. The OES sensor 270 may be a probe positioned relative the plasma jet stream to obtain spectral data at different regions of the plasma jet stream 240.

The optional magnetic field generator 206 includes an electromagnet 280 that is coupled to a power regulator 282 to generate a magnetic field 284 around the target area 202. The strength of the magnetic field 284 may be controlled by the controller 208 and the parameters of the magnetic field may constitute additional inputs to train the diagnostic neural network module 112.

Specifically, for the thermal equilibrium plasmas, the diagnostic neural network module 112 can output the mean gas temperature, or the energy distribution function shared by all species in the plasma jet 240 in FIG. 2. Based on the diagnostic method explained below, the algorithm may be expanded to control and optimize the chemical composition in any plasma. Receiving the chemical composition and the distribution function from the trained diagnostic neural network module 112, the control neural network module 114 can thus provide a set of optimized control parameters including the compositions of input gases and discharge voltage. The control parameters such as the discharge voltage and flow of input gases (e.g., He, N2, O2, and H2O) are input into the controller 208 of the plasma generator 116 to adjust the chemical composition of the plasma jet 240.

In this example, the control parameters output from the control neural network module 114 include a set of multipliers to modify the gas input densities for the plasma generator module 116. The gas input densities are defined as the number of molecules per unit volume. Therefore, such a set of multipliers can be applied to the flow rate of each gas supply 252, 254 and 256 in FIG. 2 via control of the valves 262, 264, and 266. The voltage output by the voltage supply 212 may also be determined for the composition. Such a diagnostic-optimization method merely consists of several matrix multiplications, with each matrix at the scale of hundreds by hundreds. The diagnostic may be performed by any suitable processor in a millisecond time frame to allow for real-time diagnostics in relation to the diagnostic neural network as well as the control neural network.

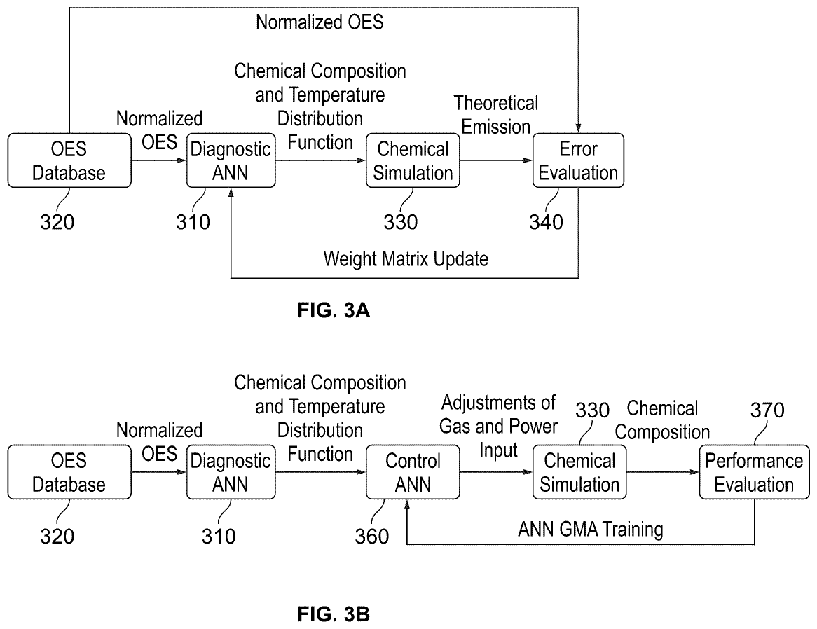

FIG. 3 shows the process of training the diagnostic neural network module 114 in FIG. 1. In this example, the neural network module 114 includes a diagnostic neural network 310 that requires training in order to accurately output the different species composition in the plasma jet. An OES database 320 includes a training data set that includes spectral data detected by the OES sensor 270 and normalized by the OES measurement module 110 for different plasma jets generated with different gas compositions and voltages. In each training iteration, the diagnostic neural network 310 is trained with a randomly selected normalized OES dataset from the training dataset in the OFS database 320. The training dataset includes OES data such as spectral data detected by the OES sensor 270 and normalized by the OES measurement module 110 for different plasma jets generated with different gas compositions and voltages.

The training dataset allows for the neural network 310 to be trained to determine the actual species composition based on the spectral data obtained from the OES sensor 270. Thus, a novel gradual-mutation algorithm (GMA) which is a variation of regular evolutionary algorithms is tailored for the training of the diagnostic neural network 310. The GMA is a type of evolutionary algorithm. A regular way to update the weights of neural networks is the backward propagation based on the error gradients. However, considering the complexity of plasma chemistry, a derivation of error gradients is not available. Therefore, an evolutionary algorithm is used. At the beginning of each iteration of the training, an input is picked up randomly from the training database. Next, multiple mutational neural networks are created based on the original neural network. Each weight in a mutational neural network equals the corresponding weight value in the original neural network adding a small random number. All the mutational neural networks will receive the same input, like the original neural network, and each of them will thus provide a unique output. Along with the output of the original neural network, all outputs will be evaluated, and the neural network with the lowest error will become the original neural network of the next iteration. The iteration will end when the error converges to an acceptable low value.

After feed-forward computation, the output of the diagnostic neural network 310 is fed into a 0-D chemical simulation 330 to obtain the theoretical spectral emission based on the equation:

$$\frac{\partial {n}_{p}}{\partial t} = {\sum }_{q} ( {k}_{q} {\prod }_{r} {n}_{q , r} ) \qquad ( 1 )$$

where nₚ is the density of the p^{th} species (product) determined by the summation of several rate equations indexed by q, and the q^{th} chemical reaction rate equals the rate coefficient k_{q} times the products of all the densities of its reactants n_{q,r}. The rate coefficients k_{q} can be functions of temperatures or energy of reactants, and some of them have to be specified by solving the Boltzmann equation rather than being cited from other experimental references. The results of the chemical simulation 330 are the theoretical emission from the species composition. The results are sent to an error evaluation module 340 that compares the theoretical emission to the normalized OES data from the OES database 320. Based on the feedback, the error evaluation module 340 adjusts the weights of the nodes in the diagnostic neural network 310.

In the OES database 320, the products of chemical reactions include not only chemical species but also photons emitted from the excited atoms and molecules. The photons thus provide a spectrum of theoretical emission. The spectrum is compared with the original OES input to compute the error that may be expressed as:

$${I}_{err} = \vert {I}_{ex} ( λ ) - {I}_{com} ( λ ) \vert \qquad ( 2 )$$

where Iₑᵣᵣ is the error to minimize, Iₑₓ is the OES intensity input to the diagnostic neural network 310, I_{com} is the OFS intensity computed by the diagnostic neural network 310, and 2 is the wavelength. However, only the spectrum dataset as inputs is known while the actual species composition for each case is unknown. FIG. 3B is a flow chart of the process of training a control neural network 360 for the control neural network module 114 in FIG. 1. In this example, the control neural network 360 is trained to output control parameters to generate a plasma jet with optimal amounts of a selected chemical species or set of species. As explained above the OES database 320 provides input data for a normalized OES for the trained diagnostic neural network 310. The chemical composition and temperature distribution functions output by the trained diagnostic neural network 310 from the training data set are fed into the control neural network 360.

Once the diagnostic neural network 310 is trained, the outputs of the trained diagnostic neural network are connected to the input layer of the control neural network 360 to translate the input OES data into the chemical composition and temperature information.

At the beginning of the training iteration for the control neural network 360, OES data is picked up randomly from the OES dataset. The selected OES data is sent to the trained diagnostic neural network 360 to determine the chemical composition and temperature information. This is the input to the control neural network 360. A GMA is used and all resulting mutational control neural networks share the same input and each of them provide a unique output that is the control parameters. For example, the unique outputs may include the density variation of He, N2, O2, H2O, and the discharge voltage (or input power etc. that can control the electron temperature). Next, each output will be used to modify the chemical composition and the results are fed into the OD chemical simulation. The performance evaluation module 370 adjusts the weights for the nodes of the control neural network 360 based on the simulated chemical composition and that provided by the trained diagnostic neural network. The mutational control neural network that can provide the highest desired species density becomes the original neural network of the next iteration. Once the density of the desired species converges and cannot be increased further, the training is completed.

In each training iteration, the previously trained diagnostic neural network 310 feeds the output chemical composition and other information to the control neural network 360. The control neural network 360 computes a set of control multipliers to modify the compositions. The chemical simulation results in a set of species ratios based on the modified compositions. The training of control neural network 360 by the performance evaluation module 370 is to maximize a gain function for the desired chemical species. Such a gain function, G, is expressed as:

$$G = {γ}_{2} - {γ}_{1} \qquad ( 3 )$$

In the gain function equation above, the species ratio, γ, is defined as

$$\frac{\sum {n}_{i}}{n - \sum {n}_{i}} ,$$

where nᵢ is the required species density and n is the total density. On the right-hand side of the above equation the subscript “2” represents the species ratio after the altering of plasma parameters while the subscript “1” represents the initial one. There are two ways to increase the species ratio: increasing the densities of the wanted species or decreasing other the densities of other species. This definition is more effective than the simple γ=Σnᵢ definition because the absolute concentration can be raised by increasing the treatment time.

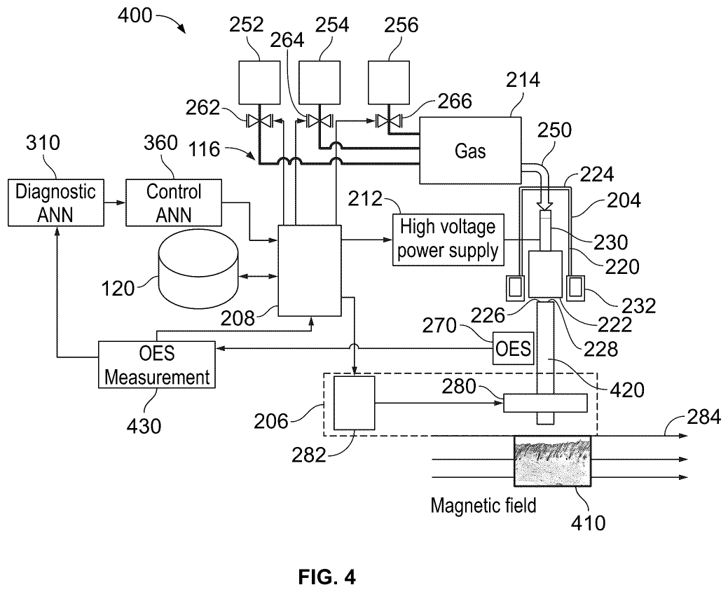

FIG. 4 is a block diagram of a plasma jet cancer treatment system 400 that generates a plasma jet based on control parameters determined by the trained control neural network 360 in real-time. The cancer treatment system 400 subjects an area of a patient 410 to a cold atmospheric plasma jet 420 and an optional magnetic field. The plasma jet 420 has a selected chemical composition that optimizes desired species to the type of treatment. For example, if the area of the patient 410 has cancerous cells, the chemical composition of the plasma jet 420 is selected to subject the cancerous cells to apoptosis. The system 400 is similar to the system 200 in FIG. 2 and thus like elements are labeled with like reference numerals in FIG. 2. In this example the controller 208 controls the mixture and strength of the plasma jet 420 and may be adjusted in real-time from the output of the trained control neural network 360 to account for environmental and other factors to produce a desired composition of species in the plasma jet 420.

In this example, the controller 208 controls different three gas sources that provide the noble gas, helium, and oxygen and nitrogen gasses to form the composition in the gas supply 214. In this manner, the controller 208 may select the gas composition from the gas supply 214 based on initial preconfigured weights from the database 120 for loading into the neural network 360. The initial preconfigured weights reflect a trained control neural network model for a desired chemical species composition. The outputs of the trained control neural network 360 in FIG. 3B allow for real-time adjustment of the plasma jet to produce the desired chemical species. It is to be understood that there may be multiple gas sources with different gasses to be combined and controlled via the controller 208 and the system 100 in FIG. 1. As will be explained, the composition may be determined by the trained control neural network 360 for optimization of selected species based on the gas type, composition, and intended effect on the patient 410. In this example, previously determined weights for the trained control neural network 360 may be stored in the database 120 and accessed when a selected species matches a previously determined species that the control neural network has been trained for. The database 120 may be managed by a separate processing device such as a server, or may be integrated into the controller 208. Alternatively, the weights for one or more trained control neural network types for different chemical species compositions may be stored in the internal memory of the controller 208.

In this example, the plasma jet 420 is optimized to have a high ionization as it exits the syringe 220. Accordingly, the syringe 220 is preferably placed at a predetermined distance from the target cells of the patient 410 being treated. The syringe 220 allows the plasma to be targeted at desired cancer cells in the skin to selectively eradicate the cancerous cells and reduce tumor size. The syringe 220 may be utilized, for instance, to treat any cancer type that is close to the skin and can be applied without surgery, such as breast, colon, lung, bladder, or oral. With surgery, the system 200 may be applied to any tumor. In this example, a distance between the central electrode 230 and the ring electrode 232 of 5-10 mm. The plasma preferably has a density of about 3×10 1 to 9×10 1-cm³, such as discussed in “Temporary-resolved measurement of electron density in small atmospheric plasmas,” to Shashurin et al, Applied Physics Letters 96, 171502 (2010), which is hereby incorporated by reference. At the predetermined distance, the plasma will have diffused to a desirable level. However, the intensity of the plasma will continue to decrease as the target area is moved further from the syringe 220, and the plasma will be essentially entirely dissipated at a distance of 5 cm from the syringe 220 in this example. The plasma is well collimated the entire length up to about 5 cm from the syringe 220. The plasma jet stream 420 is discontinuous and represents a series of propagating plasma bundles. As will be explained below, the control neural network 360 may be employed to provide control parameters to the controller 208 in real-time to adjust the plasma jet 240 to output different species depending on the treatment desired.

The actual chemical composition of the plasma jet stream 420 may be sensed by the optical emission spectroscopy sensor 270 that provides feedback data to an OES measurement module 430 similar to the OES measurement module 110 in FIG. 1. The feedback data is fed into the trained diagnostic neural network 310. The trained diagnostic neural network 310 outputs the chemical composition of the plasma jet 420 in real-time to the trained control neural network 360 to allow output of control parameters to the controller 208. The spectral data output by the OES measurement module may be stored as additional data to update the training data sets for both the diagnostic neural network module 112 and the control neural network module 114 in FIG. 1.

In this example, the plasma jet 420 may be used in combination with the optional magnetic field 284 to eradicate cancerous cells but does not have a significant effect on healthy cells surrounding the cancerous cells. The magnetic field 284 and the movement of the syringe 220 allows the in vivo treatment to be focused to specific areas on the patient 410 having high concentrations of cancerous cells. The specific types reactive species in the plasma jet 420 may be tailored to provide the most effective composition to treat different types of cancer cells.

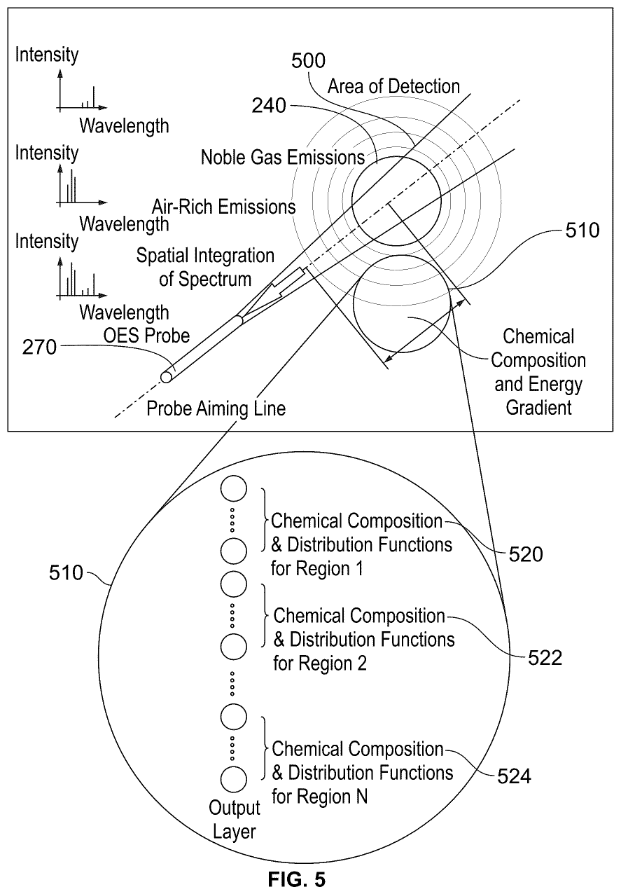

The example diagnostic algorithm is capable to spatially resolve the input OES data during the training process. FIG. 5 shows the physical collection of data by the OES sensor 270 in FIG. 2 for purposes of collecting output data for training the diagnostic neural network module 112. The OES sensor 270 outputs data to the OES measurement module 110 in FIG. 1. In this example, the OES sensor 270 is pointing at an atmospheric plasma exposure such as the plasma jet 240 in the open air. The OES sensor 270 collects and integrates all the spectral emissions from an area of detection 500. Thus, the OES sensor 270 collects data on noble gas emissions, air rich emissions, and the spatial integration of the spectrum to determine the chemical composition and energy gradient in the area of detection 500. In such a detection area 500, there are gradients of chemical compositions and temperatures/energies of those species. If such gradients are discretized into consecutive spatial regions, each region emits a different spectrum. In this example, the chemical compositions and energy gradient output layer of the diagnostic neural network 310 may be separated for separate regions 520, 522, and 524 of the area of detection 500. In this example, there are three regions, but the number of regions may be selected based on the change in the composition of the plasma jet 240 in the region. The regions may thus be selected axially or radially in the area of the plasma jet 240. The data from each region may be collected by positioning the OES probe 270 relative to the region of interest. The example diagnostic neural network can also discretize the results to provide a spatial distribution of the chemical compositions. For example, a spatially integrated OES dataset may be input to the diagnostic neural network which has been designed to provide a spatial resolution of 5 regions. The output of such a diagnostic neural network will be 5 sets of chemical compositions and temperatures. Each set of results will be tested in a OD chemical simulation and provide a theoretical OES. The final theoretical OES result of the example diagnostic neural network is the summation of 5 theoretical OES data. The final theoretical OES data will be compared with the input OES data to find the error for training diagnostic neural network.

For example, a helium-rich region at the center of the plasma jet 240 should emit helium peaks while the air-rich region surrounding the plasma jet 240 might produce the second positive system (SPS) of N₂. The resulting OES signal is the superposition of all the emissions from these regions as shown in FIG. 5. However, such a superposition may either not have a proper chemical species solution or lead to wrong solutions. Therefore, the reverse computation must be achieved with a spatial resolution. In FIG. 5, the discretization of such a chemical composition and energy gradient in the output layer of the diagnostic neural network 310 is shown.

When the output of the diagnostic neural network 310 is discretized into N regions, the actual neuron number of the output layer equals the neuron number for each region multiplied by N. The neurons of each region contain the full information achieved by the diagnostic neural network for that region, such as the densities of all the species and the temperatures or even distribution functions. Each region passes through the chemical simulation independently to produce a spectrum for the region, and the final spectrum computed for error estimation is the superposition of these spectra acquired from the chemical simulation.

In one example of the diagnostics of plasma chemistry, 900 OES datasets collected from the experimental measurements of a helium-guided cold atmospheric plasma jet with nitrogen and oxygen reactive species generated by the test system 200 were employed. 700 of the datasets were used for the training of the diagnosis and control neural networks 310 and 360, and the other 200 data sets of spectra were used for testing.

The example OES dataset is made by collecting the raw OES data measured from the plasma jet with different helium flow rates and discharge voltages. OES data was also measured from different positions on the plasma jet relative to the OES sensor. First, all the peaks in each spectrum data were normalized with respect to the intensity of 337.13 nm, which is an iconic peak of the cold atmospheric plasma. Next, five normalized peak intensities are collected from each spectrum. The five normalized peak intensities are 308.9 nm OH, 391.44 nm N₂⁺, 706.52 nm He, and 777.4 nm O.

The diagnostic neural network 310 in this example includes 10 fully connected hidden layers which contain 250 and 500 neurons in the last two layers and 100 neurons in each layer before them. The input layer has 101 neurons. 100 neurons of the input layer describe the input OES spectrum. An extra neuron is a bias unit that always equals 1. Adding a bias unit is a very commonly used strategy to design a neural network to improve the flexibility of the neural network fitting. The α value is 0.01 for the Leaky-ReLU activation function applied for all neurons. The mutation range is updated as R_{M}←R_{M}ζ/10, where R_{M} is the mutation range with an initial value set to 1%, and ζ is the counter of accumulative “the original weight matrix is the best version” event. The value of ζ is reset to unity once a better mutated weight matrix is found other than the original version. The volume in the action space to search for a better mutation keeps increasing when there is no update to the weight matrix. The chemical simulation computes the evolution of species densities with the time step dt set to 20 ns and the chemical reaction list with rates can be found in the supplemental materials.

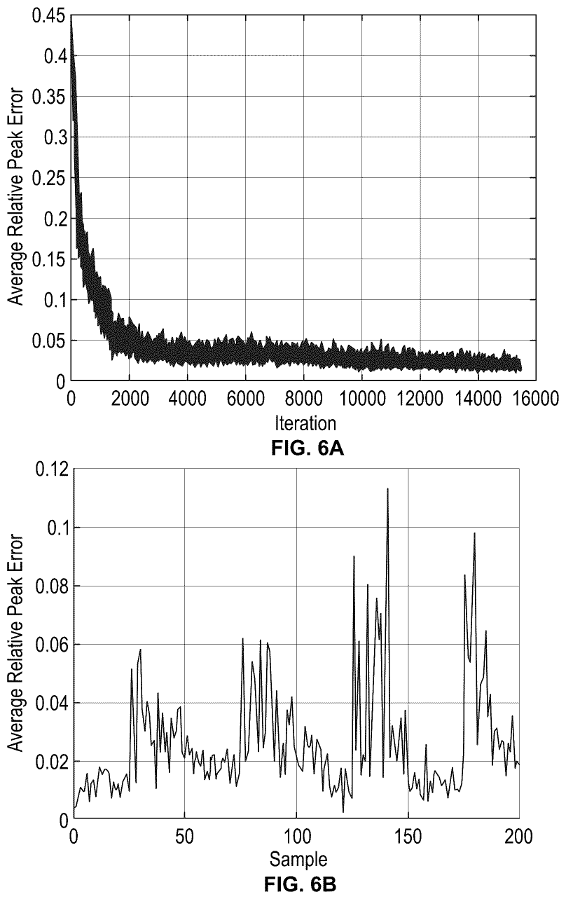

FIG. 6A shows the error convergence during the example training. FIG. 6A shows a plot of the average relative peak error and the number of iterations. Five iconic OES peaks are selected to evaluate the error. The relative peak error refers to the average error of the five selected OES peaks where the peak of 337.13 nm serves as the normalization reference. The average error of these peaks is more than 50% at the beginning iteration, and after 15,449 iterations, each peak error is lower than 3%. To strictly quantify the error, the remaining 200 samples of the OES data are input into the diagnostic neural network module 112, and the 0-D chemical simulation thus computes the photon numbers of the five emissions. A comparison of all the peaks is unnecessary because the relative lifetime (emission rate) between certain excited species is constant. The peaks are normalized (shown as the ratio) with respect to the 337.13 nm peak. The error of an iteration is the average error of the 5 peaks from the original neural network.

FIG. 6B shows the errors of the 200 testing results for the average relative peak error versus the specific sample from the test data set being processed by the trained diagnostic neural network 310. The average value of the average relative peak error for 200 testing results have a 2.608% value.

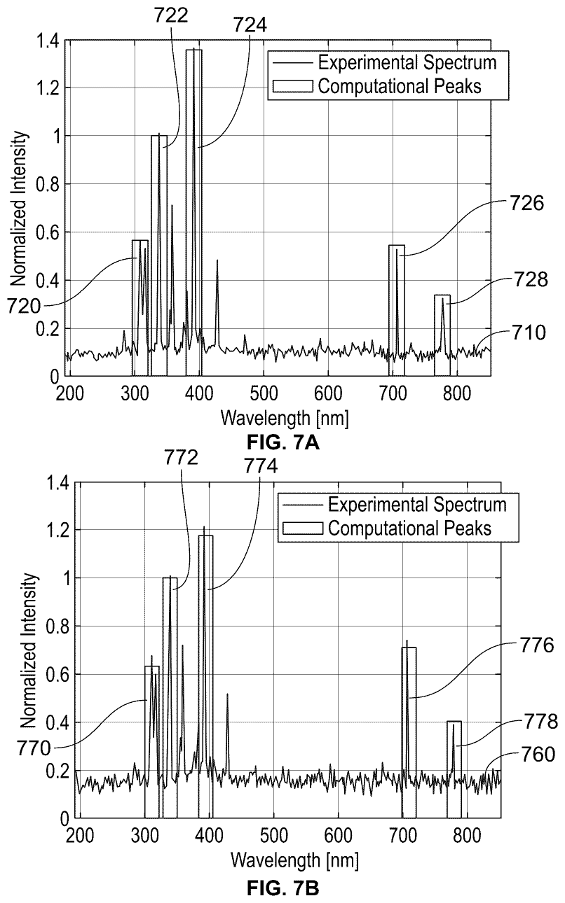

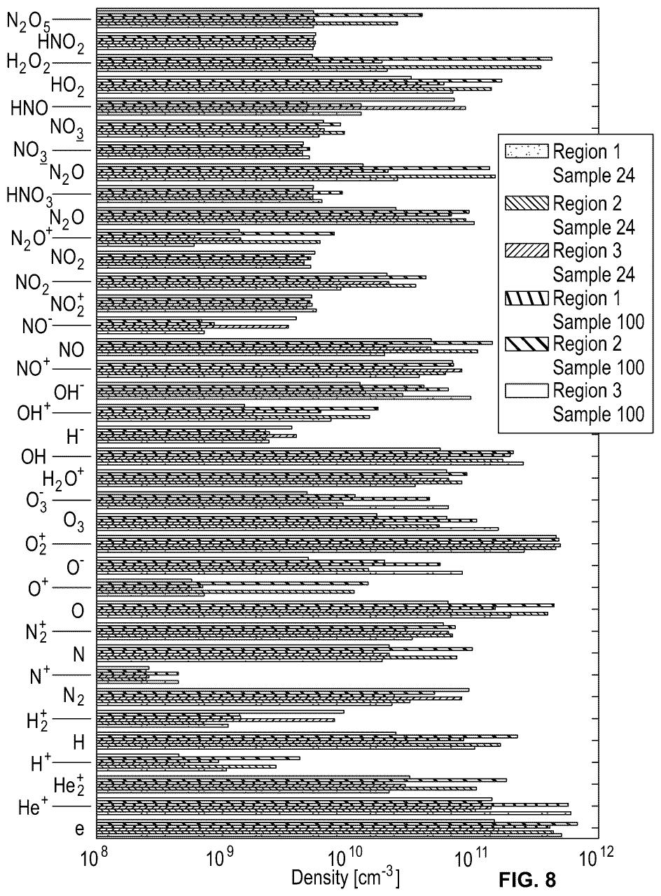

Two of the testing samples in FIG. 6B were selected arbitrarily to show the error visually in FIGS. 7A and 7B. FIG. 7A shows the normalized intensity against different wavelengths of the sample 24 in FIG. 6B and FIG. 7B shows the normalized intensity against different wavelengths of the sample 100 in FIG. 6B. The samples 24 and 100 are taken from different regions of the experimental plasma jet. A trace 710 represents an experimental spectrum in FIG. 7A for sample 24 generated from the OES data. The computational peaks and corresponding wavelengths are shown in bars 720, 722, 724, 726, and 728 in FIG. 7A. A trace 760 is an experimental spectrum in FIG. 7B for sample 100 generated from the OES data. The computational peaks and corresponding wavelengths are shown in bars 770, 772, 774, 776, and 778 in FIG. 7B.

As shown in FIGS. 7A and 7B, all the computed peaks agree well with the experimental peaks while a significant difference between sample 24 and sample 100 exists. For example, there is a height difference of the 706 nm peaks in FIG. 7A (726) and FIG. 7B (766). This difference implies the He-Air ratio. An emission next to the plasma jet nozzle should provide a high He-Air ratio while the jet tip has a lower value. Also, the gas flow rate can alter the He-Air ratio. This shows that the diagnostic neural network 310 is well trained to flexibly predict the plasma chemical composition with a variety of chemical conditions.

FIG. 8 is a chart that shows the detailed results of the predicted chemical compositions of these two cases output from the trained diagnostic neural network 310. Thus, FIG. 8 shows the density of various species (e.g., N2O5) of hydrogen, nitrogen and oxygen represented by various bars. Each of the bars for the respective species are coded to differentiate which one of three regions in the experimental plasma jet shown in FIG. 5 the species was measured and which of the two samples (24 and 100 from FIGS. 6A-6B) the species was taken from.

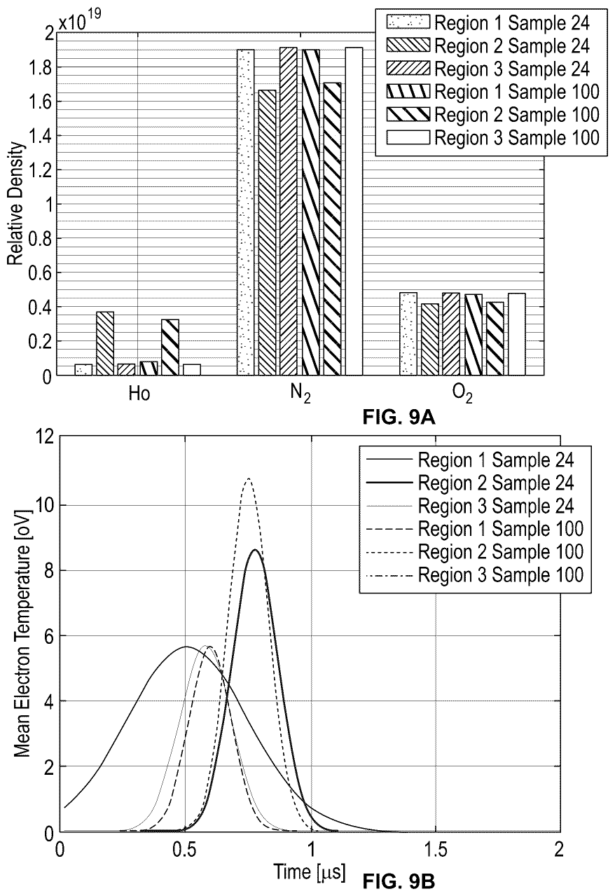

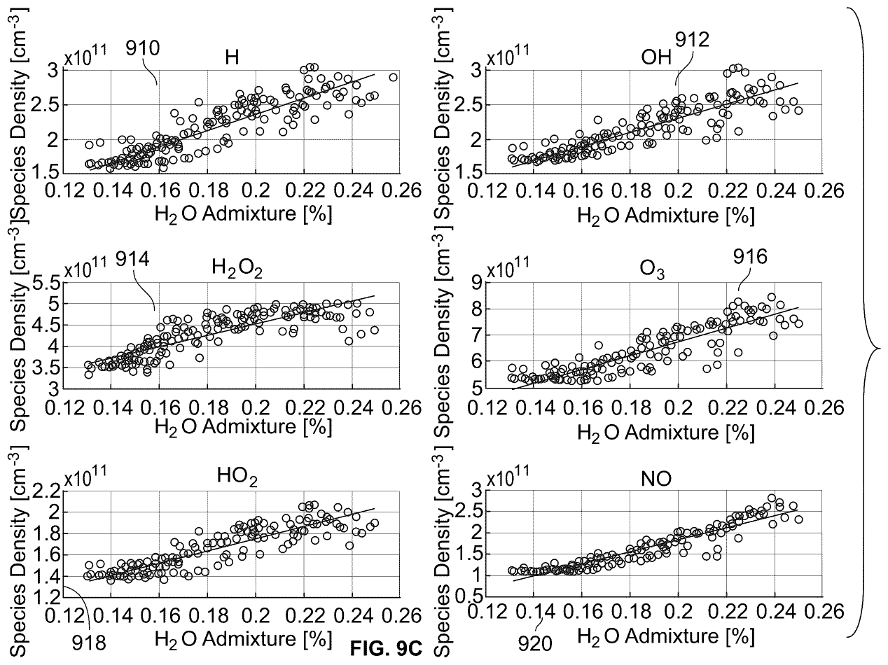

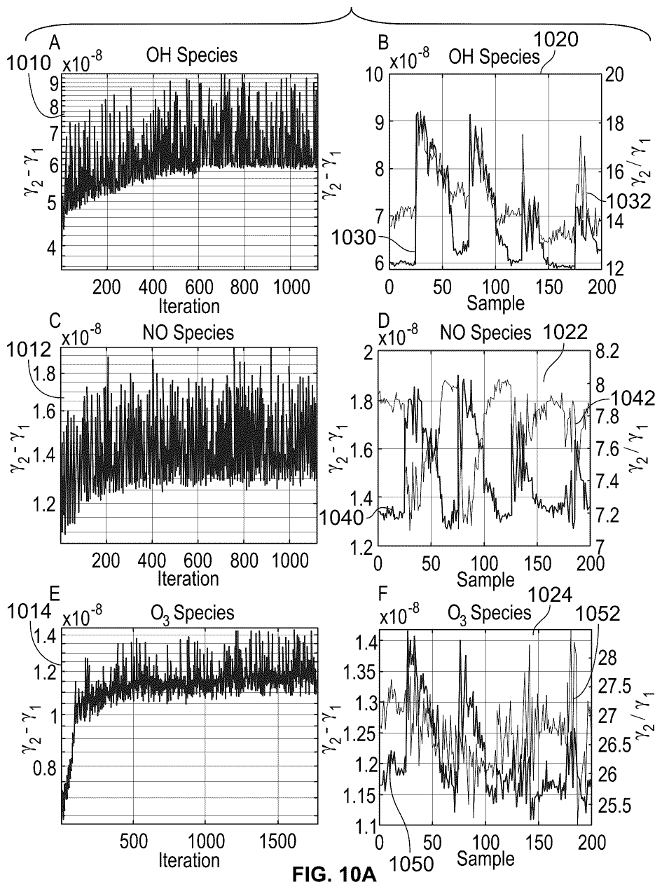

FIG. 9A shows a graph that plots the prediction of spatial resolution for different species. FIG. 9A plots the relative density of the different helium to air ratio distribution for both samples 24 and 100 from FIGS. 6A-6B in each of the three regions. FIG. 9B shows a distribution plot for the two samples of spatially resolved mean electron temperature over time. FIG. 9C shows a series of plots 910, 912, 914, 916, 918, and 920 for species densities for reactive species versus the H2O admixture. FIG. 9C shows a first plot 910 of species density of hydrogen (H) versus the H2O admixture percentage. The plot 912 shows the species density of OH versus the H2O admixture percentage. The plot 914 shows the species density of H2O2 versus the H2O admixture percentage. The plot 916 shows the species density of O3 versus the H2O admixture percentage. The plot 918 shows the species density of HO2 versus the H2O) admixture percentage. The plot 920 shows the species density of NO versus the H2O admixture percentage. The plots in FIG. 9C are combined with FIG. 9A. The measured helium to air ratio is used to double check the machine learning results. The results may be double checked with other publications of CAP chemistry using the data from FIGS. 9A-9C.

For example, in FIG. 9A, both sample 24 and sample 100 show a higher oxygen mixture in region 1 and region 3 relative to the oxygen mixture in region 2. Therefore, region 2 is the helium-rich region located in the center of the helium-guided plasma jet 240 in the open air. Region 1 and Region 3 are located between Region 2 and the surrounding air and have a lower helium-air ratio. The densities of H, N, NO, NO2, HO2, and H2O2 are reversely proportional to the oxygen admixture, while the values of OH and O3 are non-monotonic.

As shown in FIG. 8, the densities of H, N, NO, NO2, HO2, and H2O2 are higher in the oxygen-low Region 2, and no such monotonic relations for OH and O3, which agrees well with known simulations. The H2 density is the highest in Region 3 but the lowest in Region 1 with the same oxygen level of Region 3. Considering the mean electron temperature shown in FIG. 9B where the peak of Region 1 is wider than the one of Region 3, the H2 density depends on not only the oxygen admixture but also the electron energy. The OH density is higher in the high-energy Region 1 compared with Region 3. This also agrees with LIF measurement results. Similarly, the reversely proportional relationship between NO2 and air indicated in FIG. 8 can also be found in known analysis, along with the non-monotonic relationship between electron density and air admixture. Many other chemical features can be checked, such as the monotonic relationships between O and O2 admixtures. For example, FIG. 9A shows that lower O2 is present in Region 2 than in Regions 1 and 3. This is the opposite to the relation of O shown in FIG. 8. This shows a reversely proportional relation between O and O2 that corresponds to known results. Moreover, as shown in FIG. 9A, the helium densities are much lower than the air species densities (O2 and N2). This means that the OES probe 270 is pointing at the tip of CAP jet 240 where the helium-air ratio is low. In contrast, in FIG. 8, the electron density is more concentrated in Region 2. This agrees with the well-known deformation of the streamer from a donut-like pattern to a bullet-like appearance during its propagation in the CAP jet 240.

Moreover, since the environmental humidity can also vary the plasma chemistry, it is important to check the machine learning predicted RONS with respect to the H2O admixtures. As shown in the plots in FIG. 9C, these species densities are proportional to the H2O admixture, which agrees with known monotonic relationships. Environmental and target-vicinity humidity effect the plasma jet, while a shielding-gas technology can be applied to avoid the environmental chemical disturbance.

Three examples of CAP applications on plasma medicine are described herein. As explained above, concentrations of different desired species input to train the control neural network 360 based on the outputs of the trained diagnostic neural network module 112 in FIG. 1. In the first example, the control neural network 360 is trained to maximize the summation of densities of OH, HO2, H2O2, and OH ions as the active pharmaceutical ingredients (API) for cancer treatments, since these species play a key role in leading cancer cells to their apoptosis. In the second example, the control neural network 114 is trained to maximize the summation of the densities of NO and its ions as the API for a wound treatment due to its effect of inflammation reduction and supports of tissue regeneration. In the third example, the goal is to maximize the summation of the densities of O3 and its ions as the API for sterilizations. The UV emissions are not considered as an API to sterilize in this general example, due to the potential of damaging carbon bonds of the short-wavelength peaks, which may damage the certain plastic surfaces and human tissue.

In these specific examples, the control neural network 360 of the neural network module 114 includes five hidden layers with 200 and 300 neurons for the last two layers and 100 each for the others. The output layer includes 106 neurons which are defined as the normalized parameter multipliers. Each neuron of the control neural network 360 input layer represents a chemical species density coming from the corresponding neuron of the output layer of the diagnostic neural network 310. The first 100 outputs are the multipliers of the temporally resolved mean electron temperature. The OD chemical simulation used to test the control neural network has a dt at 20 ns. The 100 outputs showing the multipliers of the electron temperature, <Te>, with 20 ns between each output thus means a curve of electron temperature <Te> gain over 2000 ns. The controller 208 may magnify the discharge voltage of the plasma jet following the curve. The discharge voltage is adjusted by the controller 208 every 20 ns. After 2000 ns, the controller 208 will take a new OES signal from the spectrometer 270. To ensure that the multipliers lie between 0 and 2, the next output corresponding to the overall scale is used, and the first 101 outputs are divided by half of the maximum. The next four outputs from the 102nd to the 105^{th} outputs are the multipliers for the densities of N2, O2, H2O, and He. Similarly, the additional 106th output is also utilized for normalization.

After the multipliers are applied on the four gas densities, the values are recomputed to ensure that their summation agrees with the total particle number under the atmospheric and room-temperature conditions, but the newly acquired ratio of N2:O2:H2O:He is unchanged. Next, the modified chemical composition and temperatures is sent to the chemical simulation. The simulation thus computes a chemical composition variation in a period to evaluate the error.

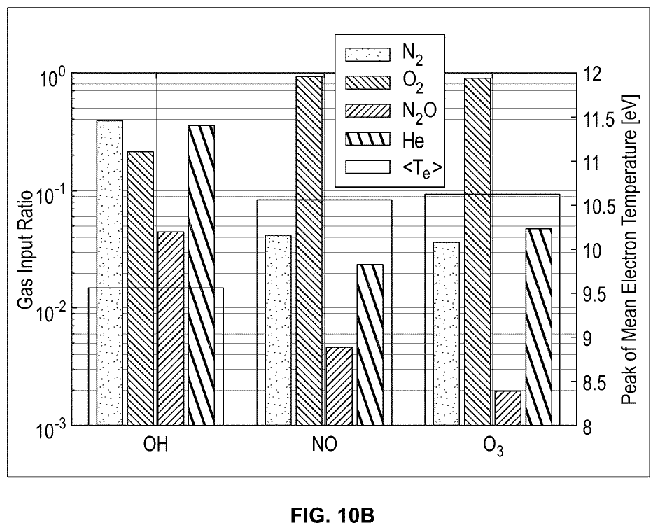

An example of the evolutions of the function G (gain for a specific species) for training and testing of the control neural network 360 in FIG. 4A are shown in FIGS. 10A-10B. The gain values of the desired species for three cases (OH, NO, and O3) after the training are at about 10⁻⁸. After the gain values are set, the additional 200 samples were tested against the trained control neural network 360.

A graph 1010 in FIG. 10A shows the convergence of gain function of OH species including OH, HO2, H2O2, and their ions over training iterations for each of the three examples. A graph 1012 shows the convergence of gain function of NO and its ions over training iterations. A graph 1014 shows the convergence of gain function of O3 and its ions over training iterations. Corresponding graphs 1020, 1022, and 1022 show the convergence of the gain function and the ratio of the gain function for each of the three cases.

The corresponding graph 1020 for the OH species plots the gain in a first trace 1030 and a gain ratio in a second trace 1032 of each of the 200 test samples using the trained control neural network 360. The corresponding graph 1022 for the NO species plots the gain in a first trace 1040 and a gain ratio in a second trace 1042 of each of the test 200 samples using the trained artificial control neural network 360. The corresponding graph 1024 for the O3 species plots the gain in a first trace 1050 and a gain ratio in a second trace 1052 for each of the 200 test samples using the trained artificial control neural network 360. As shown in the graphs 1022, 1024, and 1026 the average increments of γ are about 15, 7.5, and 27 times for the case of OH, NO, and O3, respectively.

FIG. 10B shows the resulting outputs of the trained control neural network 360 for each of the three examples. The outputs of the control neural network 360 suggest different gas compositions and peak mean electron temperatures to maximize the composition of active pharmaceutical ingredients of the three training examples. Thus, as shown in FIG. 10B, the control neural network 360 suggests gas composition and peak mean electron temperature to maximize the composition of active pharmaceutical ingredients of the three training cases. The combination of N2, O2, H2O, and He are shown for the three examples suggested by the control neural network 360 to achieve the optimizations. These values are the averages over the 200 testing samples.

As shown in FIG. 10B, to maximize the gain (G) of the desired OH species for the cancer therapy, the example trained control neural network 360 suggests approximately a combination of 39.61% of N2, 21.15% of O2, 4.44% of H2O, and 35.35% of He to produce the desired OH species. The chemical composition of H2O is extremely high compared with regular humid air. For example, a 4% molar fraction of H2O in a 300 K and 1 atm air is almost 100% relative humidity, which means the saturation of vapor and about to rain. However, it is still practical to achieve such an H2O composition, since the gas input can be a mixture including H2O-droplet aerosols rather than the H2O vapor in the air. Based on the ratios, the controller 208 in FIG. 4 may control the valves 262, 264, and 266 to obtain the desired ratios of N2, O2, and He. The controller 208 also controls the voltage output by the voltage supply 212 to obtain the desired mean electron temperature.

To maximize the G of the NO species and the G of the O3 species for the wound treatments and sterilizations, a high composition of O2 is required. The values are 93.05% of O2 for the NO species and 91.38% of O2 for the O3 species. In the example of OH optimization, the O2 composition is low since H2O is the main oxygen element provider. The gas composition between the NO and O3 examples is close. However, NO requires more N2 and H2O inputs but less He than the O3 example. On the other hand, the peak of mean electron temperature denoted as <Te> for these three cases is shown in FIG. 10B. The values of the electron temperature for the NO and O3 cases are much higher than the ones for OH optimization. Considering the high O2 compositions for the NO and O3 cases and the high negativity of the oxygen element, a lower electron temperature makes a higher loss of electron due to the attachments. Therefore, higher electron temperatures are required when extra O2 is added. Note that these examples are controls of the CAP jet, and the peak mean electron temperatures shown in FIG. 10B are the mean electron temperature values of streamers passing through the area of OES probe detection. For a mean electron temperature spatially averaged over an entire CAP jet, the value will at around 3-5 eV as measured in previously known references.

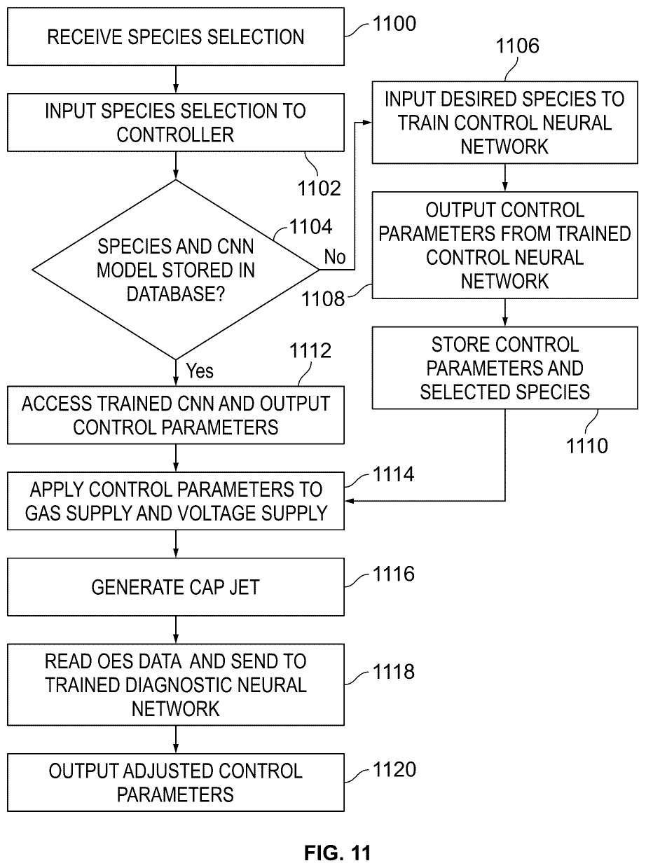

FIG. 11 is a flow diagram of a routine executed by the controller 208 to control the composition of a cold atmospheric plasma jet. The flow diagram in FIG. 11 is representative of an example routine implementable by machine-readable instructions for the controller 208 in FIG. 4. In this example, the machine-readable instructions comprise an algorithm for execution by (a) a processor; (b) a controller; and/or (c) one or more other suitable processing device(s). The algorithm may be embodied in software stored on tangible media such as flash memory, CD-ROM, floppy disk, hard drive, solid-state drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application-specific integrated circuit (ASIC), a programmable logic device (PLD), a field-programmable logic device (FPLD), a field-programmable gate array (FPGA), discrete logic, etc.). For example, any or all of the components of the interfaces can be implemented by software, hardware, and/or firmware. Also, some or all of the machine-readable instructions represented by the flowcharts may be implemented manually. Further, although the example algorithm is described with reference to the flowchart illustrated in FIG. 11, persons of ordinary skill in the art will readily appreciate that many other methods of implementing the example machine-readable instructions may alternatively be used. For example, the order of execution of the blocks may be changed, and/or some of the blocks described may be changed, eliminated, or combined.

The controller 208 first receives a desired species selection for a treatment purpose (1100). As explained above, a selection could be high densities of OH, HO2, H2O2, and OH ions for cancer treatment, another selection could be to maximize NO and associated ion densities for wound treatment or increased densities of O3 and its ions for sterilization. The desired species are then input to the controller 208 (1102). The controller 208 determines whether a trained model for the selected species is stored in the database 120 as the result of previously training the control neural network 360 (1104). If a trained model for the selected species are not stored in the database 120, the controller inputs the selected species to train the control neural network 360 (1106). Once, the control neural network 360 is trained to optimize the selected species, the control neural network 360 outputs the appropriate control parameters (1108). The output control parameters and selected species are then stored in the database 120 for future use (1110).

If the selected species match species that are stored in the database 120, the controller 208 loads the trained model from the database 120 to control neural network and outputs appropriate control parameters (1112). Once the control parameters are loaded from either the newly trained control neural network 360 or the model from the database 120, the controller 208 applies the control parameters to the voltage supply and the gas supplies (1114). The cold atmospheric plasma jet is generated and directed toward the target area (1116). The controller 208 also activates the OES sensor 270 and the measured spectral data is sent to the trained diagnostic neural network 310 (1118). The output chemical composition from the diagnostic neural network 310 is input into the control neural network 360. Based on the input, the control neural network 360 outputs adjustments to the control parameters in real-time (1120). The controller 208 applies the adjusted control parameters to the generation of the plasma jet 240. The spectral data may also be added to the diagnostic training dataset in the database 120.

The above described system is a real-time diagnostic of chemical composition and a control method for generating a cold atmospheric plasma jet. Taking the real-time and passive measurement of the spontaneous emissions from the plasma jet as a continuous input, the diagnostic neural network module 112 can reversely compute the chemical compositions and temperatures behind such spectra. An example of helium-guided CAP chemistry with a variety of O2 and H2O admixtures demonstrates the system. The trends of species variations agree well with previously measured data. The example system uses simple hardware but relies more on computation to provide more complex analysis. The acquisition of chemical compositions from spontaneous emission spectroscopy is achieved through machine learning.

Applying this method to plasma medicine, the plasma treatment can be immune to external disturbance, such as the sudden change of the target position leading to an extra gas flow and a different electromagnetic boundary condition for the electron temperature. Therefore, the real-time control system makes plasma therapy automatically adaptive to dynamic situations. In other words, self-adaptive plasma chemistry is the core of intelligent plasma medicine. Second, a single CAP generator can be used for several different plasma treatments. Each type of plasma therapy requires unique plasma chemistry and the machine learning networks are trained independently. These weight matrices can be stored in a single microcontroller chip controlling the same plasma generator where the working mode can be switched among the types of plasma therapy. Alternatively, the weight matrices may be stored in accessible memory to set the controller. For example, the CAP generator can be switched to optimize the OH family aiming at a certain cancer cell line, while the same hardware can also be switched to maximize O3 for solid surface sterilization.

Intelligent plasma therapy can bypass the diagnostics of plasma chemistry but map the space of a target condition and the space of the plasma control inputs. However, such a mapping is established in specific situations. For example, due to the different responses from different cancer cell lines, each type of cancer requires stand-alone training. Alternately, focusing on the self-adaptive plasma chemistry (composition optimization) can lead to a broad-spectrum therapy. Also, sometimes the target feedback signals may not be available, such as the real-time measurement and prediction of in vivo situations of cell/bacteria/virus.

Finally, this method may be applied to fields outside the biomedical field, that depends on the spontaneous emissions. All plasmas with certain species temperatures should contain excited species due to the collisions. Thus, the principles disclosed herein may be applied to all types of plasmas, from a fusion reactor to the plasma-enhanced chemical vapor depositions (PECVD) and etching. For example, in PECVD processing, issues such as the deposition rates, the growth mechanisms, and the deposition accuracy on complex geometries may be solved using machine-learning. Meanwhile, OES is a commonly used diagnostic in the community of PECVD and nanomaterials. Therefore, it is easy to apply this general method to other plasma communities, especially the plasma-based material synthesis.

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Numerous changes to the disclosed embodiments can be made in accordance with the disclosure herein, without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

1. A system for treatment of a target area, comprising:
a plasma jet emitter emitting a plasma jet at the target area;
a gas source providing a gas composition to the plasma jet emitter;
a voltage source coupled to an electrode in the plasma jet emitter;
a controller coupled to the gas source and the voltage source to control the plasma jet emitter to generate a plasma jet having a desired chemical composition via control parameters for the gas source and the voltage source;
an optical emission spectroscopy sensor to detect spectral data from the plasma jet;
a diagnostic neural network module trained to output chemical compositions and energy distribution of gasses of the gas source from an input of spectral data from the optical emission spectroscopy sensor, wherein the detected spectral data is input to the diagnostic neural network; and
a control neural network module coupled to the controller and the diagnostic neural network, the control neural network having an input of the chemical compositions and energy distribution of the gasses of the gas source output by the diagnostic neural network and an output of the control parameters, the control neural network trained via chemical compositions output from the diagnostic neural network and the desired chemical composition.

2. The system of claim 1, further comprising a database coupled to the controller, the database storing trained control neural network data for a plurality of chemical compositions including the desired chemical composition.

3. The system of claim 1, wherein the chemical composition of the plasma jet includes at least one of a reactive oxygen or nitrogen species and wherein the gasses of the gas source include a noble gas, oxygen, and nitrogen.

4. (canceled)

5. The system of claim 1, further comprising a magnetic field generator controlled by the controller and directing a magnetic field at the target area, wherein the control parameters include the strength of the magnetic field.

6. (canceled)

7. The system of claim 1, wherein the training of the diagnostic neural network includes comparison of spectral data determined by a chemical simulation of the chemical composition with the results of the chemical composition determined by the diagnostic neural network based on the spectral data.

8. The system of claim 1, wherein the training of the control neural network compares control parameters output from the control neural network and control parameters associated with a simulation of the desired composition.

9. The system of claim 1, wherein the training of the diagnostic neural network and control neural network uses a gradual mutation algorithm (GMA) to create multiple mutation neural networks providing unique outputs and selects the mutation neural network with a lowest error for a next iteration of training.

10. The system of claim 1, wherein the chemical composition maximizes the summation of densities of OH, HO2, H2O2, and OH ions as the active pharmaceutical ingredients (API) and the treatment is apoptosis of cancer cells in the target area.

11. The system of claim 1, wherein the chemical composition maximizes the summation of the densities of NO and its ions and the treatment is healing of a wound in the target area.

12. The system of claim 1, wherein the chemical composition maximizes the summation of the densities of O3 and its ions and the treatment is sterilizing the target area.

13-20. (canceled)

21. A method of generating a plasma jet having a specific chemical composition, the method comprising:
selecting a desired chemical composition for the plasma jet;
generating a plasma jet;
detecting spectral data from the plasma jet;
inputting the spectral data to a diagnostic neural network to output the chemical composition of the plasma jet, wherein the diagnostic neural network is trained by spectral data from compositions of plasma jets;
determining a set of control parameters via a control neural network trained via chemical compositions output by a diagnostic neural network; and
applying the control parameters to a gas source and a voltage generator to adjust the plasma jet via a controller; and
directing the generated plasma jet to a target area for treatment.

22. The method of claim 21, wherein the control neural network is programmed from trained control neural network data for a plurality of chemical compositions including the desired chemical composition stored in a database.

23. The method of claim 21, wherein the chemical composition of the plasma jet includes at least one of a reactive oxygen or nitrogen species and wherein the gasses of the gas source include a noble gas, oxygen, and nitrogen.

24. (canceled)

25. The method of claim 21, further comprising directing a magnetic field at the target area via a magnetic field generator controlled by the controller, wherein the control parameters include the strength of the magnetic field.

26. (canceled)

27. The method of claim 21, wherein the training of the diagnostic neural network includes comparison of spectral data determined by a chemical simulation of the chemical composition with the results of the chemical composition determined by the diagnostic neural network based on the spectral data.

28. The method of claim 21, wherein the training of the control neural network compares control parameters output from the control neural network and control parameters associated with a simulation of the desired composition.

29. The method of claim 21, wherein the training of the diagnostic neural network and control neural network uses a gradual mutation algorithm (GMA) to create multiple mutation neural networks providing unique outputs and selects the mutation neural network with a lowest error for a next iteration of training.

30. The method of claim 21, wherein the chemical composition maximizes the summation of densities of OH, HO2, H2O2, and OH ions as the active pharmaceutical ingredients (API) and the treatment is apoptosis of cancer cells in the target area.

31. The method of claim 21, wherein the chemical composition maximizes the summation of the densities of NO and its ions and the treatment is healing of a wound in the target area.

32. The method of claim 21, wherein the chemical composition maximizes the summation of the densities of O3 and its ions and the treatment is sterilizing the target area.

33-40. (canceled)
